(19) United States
(12) Patent Application Publication
(10) Pub. No.: US 2014/0085448 A1
(43) Pub. Date: Mar. 27, 2014

(54) IMAGE PROCESSING APPARATUS

(71) Applicant: Olympus Corporation, Tokyo (JP)

(72) Inventor: Motohiro MITAMURA, Tokyo (JP)

(73) Assignee: OLYMPUS CORPORATION, Tokyo (JP)

(21) Appl. No.: 14/090,046

(22) Filed: Nov. 26, 2013

Related U.S. Application Data
(63) Continuation of application No. PCT/JP2012/063609, filed on May 28, 2012.

(30) Foreign Application Priority Data
Jun. 1, 2011 (JP) 2011-123552

(51) Int. Cl. A61B 1/00

(52) U.S. Cl. CPC A61B 1/00009
USPC 348/68

(57) ABSTRACT

The invention provides an image processing apparatus including a storage unit that stores a three-dimensional image of an observation target in a subject; a projected-image generating unit that receives an image-acquisition position and an image-acquisition direction of an acquired two-dimensional superficial image of the observation target in a surface layer of the subject and that generates a two-dimensional projected image by projecting a position corresponding to the image-acquisition position of the three-dimensional image stored in the storage unit in the image-acquisition direction; and a multiplication processing unit that receives the superficial image and the projected image and generates a multiplied image by multiplying brightness values of corresponding pixels in the superficial image and the projected image.

### CROSS-REFERENCE TO RELATED APPLICATIONS

This is a continuation of International Application PCT/JP2012/063609, with an international filing date of May 28, 2012, which is hereby incorporated by reference herein in its entirety. This application claims the benefit of Japanese Patent Application No. 2011-123552, the content of which is incorporated herein by reference.

### TECHNICAL FIELD

The present invention relates to an image processing apparatus.

### BACKGROUND ART

In the related art, there is a known observation system that superposes an image formed by two-dimensionally converting a three-dimensional image of lymphatic vessels, lymph nodes, and blood vessels obtained by a CT (computed tomography) apparatus onto a two-dimensional image of lymphatic vessels and lymph nodes obtained with an endoscope (see, for example, Patent Literature 1). A CT image is suitable for observing the rough three-dimensional structure of tissue inside the body. An endoscope image is suitable for observing the detailed structure of the surface of tissue inside the body. In other words, with the system in Patent Literature 1, superficial lymphatic vessels and lymph nodes can be observed in detail while also roughly grasping the structure of lymphatic vessels, lymph nodes, and blood vessels in deep layers.

### CITATION LIST

### Patent Literature

### {PTL 1}

Japanese Unexamined Patent Application, Publication No. 2007-244746

### SUMMARY OF INVENTION

The present invention provides an image processing apparatus including a storage unit that stores a three-dimensional image of an observation target in a subject; a projected-image generating unit that receives an image-acquisition position and an image-acquisition direction of an acquired two-dimensional superficial image of the observation target in a surface layer of the subject and that generates a two-dimensional projected image by projecting a position corresponding to the image-acquisition position of the three-dimensional image stored in the storage unit in the image-acquisition direction; and a multiplication processing unit that receives the superficial image and the projected image generated by the projected-image generating unit and generates a multiplied image by multiplying brightness values of corresponding pixels in the superficial image and the projected image.

### BRIEF DESCRIPTION OF DRAWINGS

FIG. 1 is a diagram showing the overall configuration of an endoscope system provided with an image processing apparatus according to an embodiment of the present invention.

FIG. 2 is a block diagram showing the functions of [symbol] n image processing unit in FIG. 1.

FIG. 3 includes diagrams for explaining the image processing method carried out by the image processing unit in FIG. 2, where (a) shows a projected image, (b) shows a fluorescence image, (c) shows a multiplied image, (d) shows a white-light image, and (e) shows a superposed image.

### DESCRIPTION OF EMBODIMENTS

An image processing apparatus 100 according to an embodiment of the present invention will be described below with reference to the drawings.

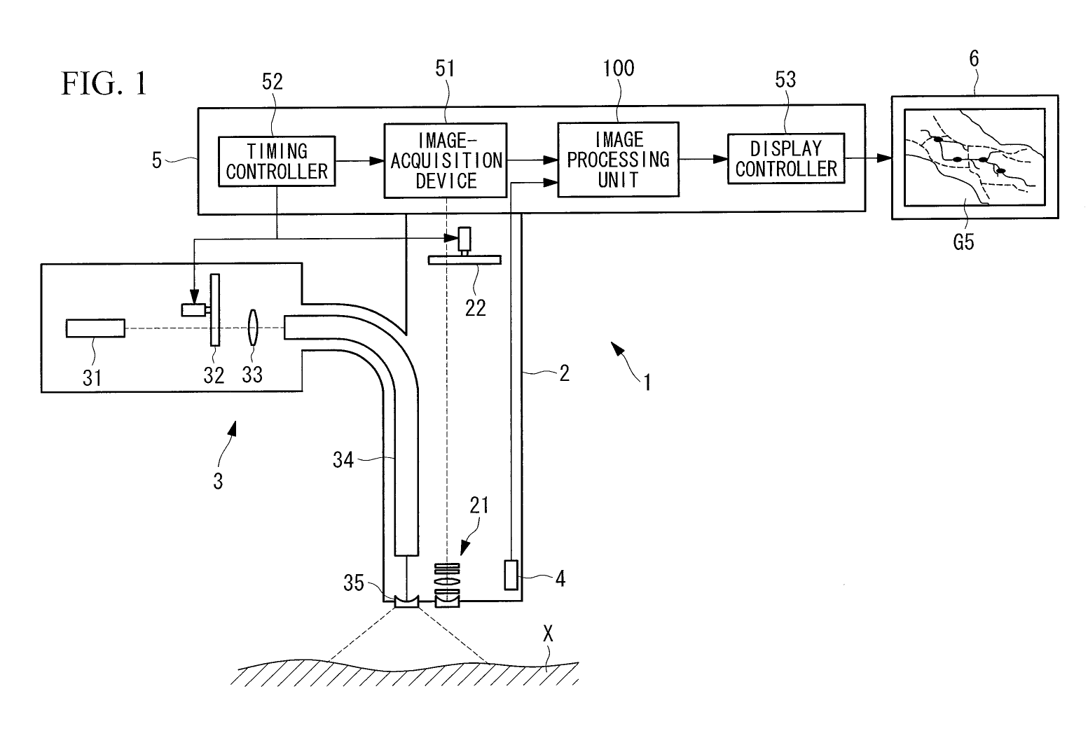

As shown in FIG. 1, the image processing apparatus 100 according to this embodiment is provided in an endoscope system 1 serving as an image processing unit (hereinafter also referred to as image processing unit 100).

The endoscope system 1 includes a long, thin inserted portion 2 having an objective optical system 21 at the distal end thereof; an illumination unit 3 that irradiates a subject X with white light and excitation light, in a time-division manner, via the inserted portion 2; a position sensor 4 provided at the distal end of the inserted portion 2; and a control unit 5 that is disposed at the proximal end of the inserted portion 2 and that generates and processes images. The image processing unit 100 in this embodiment is provided in the control unit 5.

The inserted portion 2 includes the objective optical system 21, which collects light coming from a tissue surface layer inside a living body, which serves as a subject X, and guides this light to an image-acquisition device 51 (described later), and a first filter turret 22 disposed at an intermediate position in the light path between the objective optical system 21 and the image-acquisition device 51. The first filter turret 22 includes a white-light filter that selectively transmits white light and a fluorescence filter that selectively transmits fluorescence. The light that is guided to the image-acquisition device 51 is switched between white light and fluorescence by rotating the first filter turret 22.

The illumination unit 3 includes a light source 31, a second filter turret 32 that extracts one of white light and excitation light from the light radiated from the light source 31, a coupling lens 33 that focuses the light extracted by the second filter turret 32, a light guide fiber 34 that is disposed over substantially the entire length in the longitudinal direction of the inserted portion 2, and an illumination optical system 35 that is provided at the distal end of the inserted portion 2.

The second filter turret 32 includes a white filter that selectively transmits white light (in a wavelength range of 400 nm to 740 nm) and an excitation filter that selectively transmits excitation light having a wavelength that excites a fluorescent dye. By rotating the second filter turret 32, the light that is guided in the light guide fiber 34 is switched between white light and excitation light. The light extracted by the second filter turret 32 and focused by the coupling lens 33 is guided inside the inserted portion 2 by the light guide fiber 34 and is then spread out by the illumination optical system 35 and radiated onto the subject X.

In this embodiment, by mixing indocyanine green (ICG) in the lymph fluid of the subject, a fluorescence image G2, in which the observation targets are the lymphatic vessels and lymph nodes (hereinafter, both are referred to as lymphatic vessels), is observed. ICG has excitation wavelengths from 680 nm to 780 nm and a fluorescence wavelength of 830 nm. In other words, the excitation filter transmits light having a wavelength of 680 nm to 780 nm as excitation light, and the fluorescence filter transmits light close to a wavelength of 830 nm as the fluorescence.

The position sensor 4 includes, for example, a three-axis gyro sensor and a three-axis acceleration sensor. The position sensor 4 detects changes in the position and angle, in three axial directions, from a reference position and a reference direction and sums the detected changes in each direction. By doing so, the position sensor 4 calculates the current position and current direction of the distal end of the inserted portion 2 with respect to the reference position and reference direction, in other words, the image-acquisition position and image-acquisition direction of the image acquired by the image-acquisition device 51 (described later). The reference position and reference direction of the position sensor 4 can be set to any position and direction based on an operation performed by the operator. The position sensor 4 outputs the calculated current position and current direction to a projected-image generating circuit 104 (described later) in the image processing unit 100.

The control unit 5 includes an image-acquisition device 51 that acquires the white light and fluorescence and generates image data, a timing controller 52 that switches between generating a white-light image and generating a fluorescence image, and a display controller 53 that outputs the image generated by the image processing unit 100 on a monitor 6.

The timing controller 52 has a white-light mode and a fluorescence mode. In the white-light mode, the timing controller 52 rotates the first and second filter turrets 22 and 32 so as to place the white-light filters in the light path and outputs the image data from the image-acquisition device 51 to a white-light-image generating circuit 101 (described later) in the image processing unit 100. In the fluorescence mode, the timing controller 52 rotates the first and second filter turrets 22 and 32 so as to place the excitation filter and the fluorescence filter in the light path and outputs the image data from the image-acquisition device 51 to a fluorescence-image generating circuit 102 (described later). The timing controller 52 alternately switches between these two modes at sufficiently short time intervals. By doing so, the image processing unit 100 alternately generates a white-light image G1 and a fluorescence image G2 at sufficiently short time intervals.

The display controller 53 outputs superposed images G5 on the monitor 6 at a prescribed timing in such a manner that a prescribed number of superposed images G5 (described later) in one second are displayed on the monitor 6 at constant time intervals.

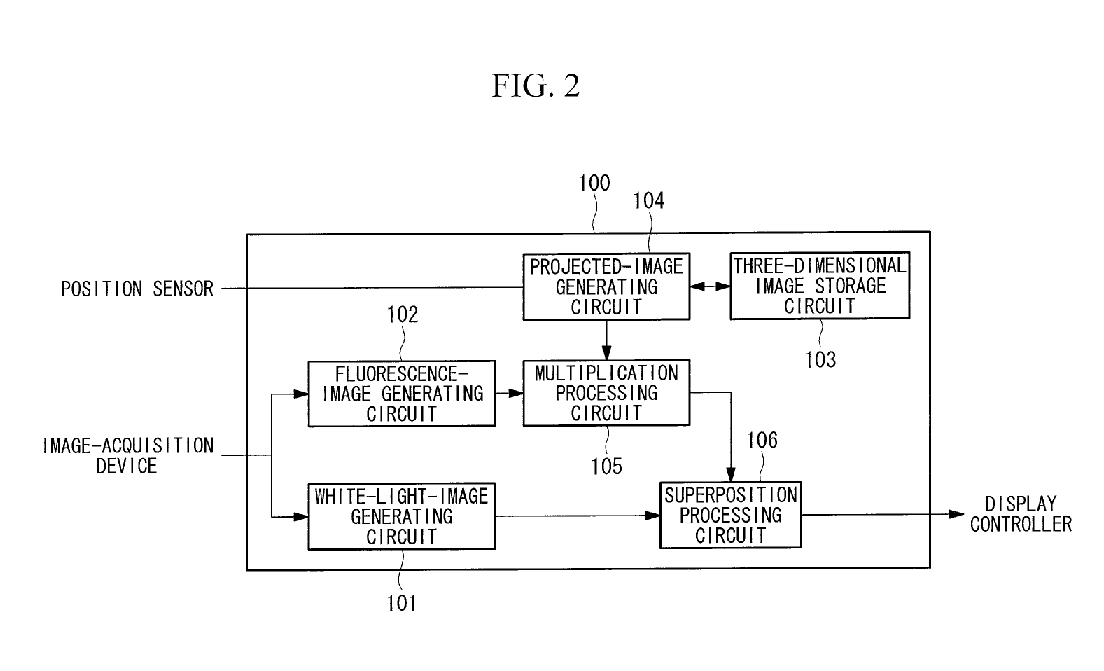

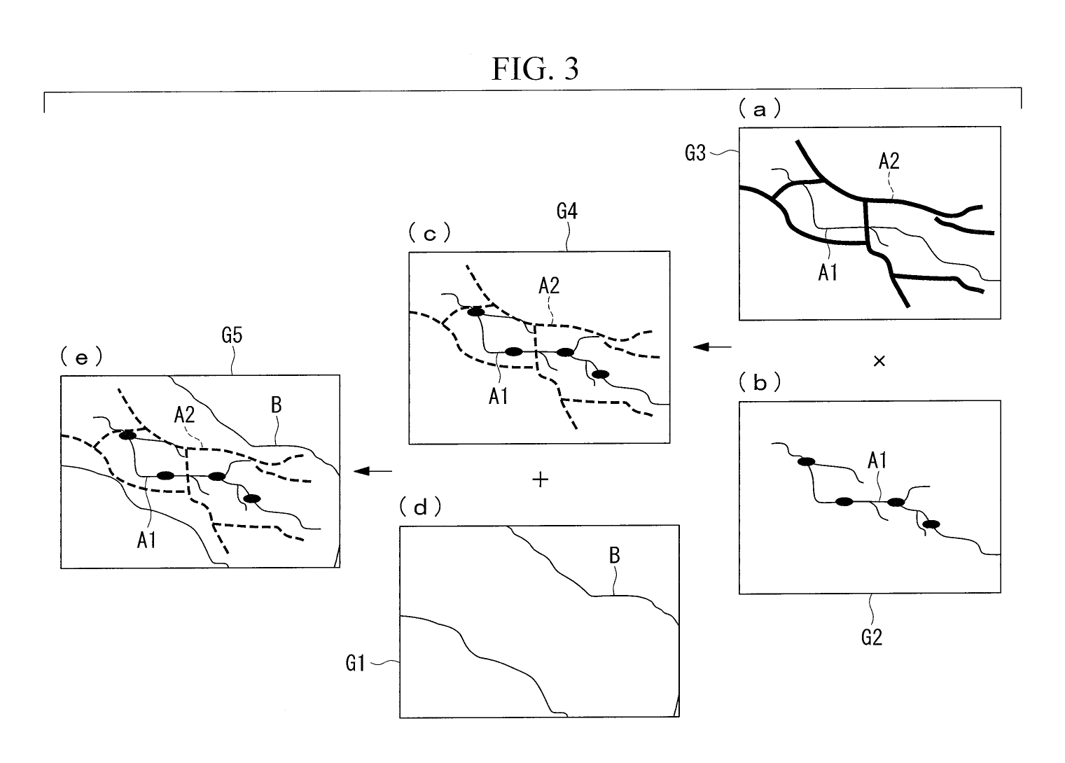

As shown in FIG. 2, the image processing unit 100 includes the white-light-image generating circuit 101, which generates the white-light image G1; the fluorescence-image generating circuit 102, which generates the fluorescence image G2; a three-dimensional image storage circuit (storage unit) 103 that records a three-dimensional image of the subject acquired by a three-dimensional observation device; a projected-image generating circuit 104 that generates a two-dimensional projected image G3 from the three-dimensional image stored in the three-dimensional image storage circuit 103; a multiplication processing circuit (multiplication processing unit) 105 that generates a multiplied image G4 by multiplying the projected image G3 and the fluorescence image G2 by a brightness value; and a superposition processing circuit (superposition processing unit) 106 that generates a superposed image G5 by superposing the multiplied image G4 on the white-light image G1. FIG. 3 is a conceptual diagram for explaining the image processing method performed by the image processing unit 100.

The white-light-image generating circuit 101 generates the white-light image G1 from the white-light image data input from the image-acquisition device 51 and outputs the generated white-light image G1 (see (d) in FIG. 3) to the superposition processing circuit 106.

The fluorescence-image generating circuit 102 generates the fluorescence image (superficial image; see (b) in FIG. 3) from the fluorescence image data input from the image-acquisition device 51 and outputs the generated fluorescence image G2 to the multiplication processing circuit 105. In the fluorescence image G2, superficial lymphatic vessels A1 in the tissue, which are the observation targets, are displayed as fluorescence regions, that is to say, as bright regions.

The three-dimensional image storage circuit 103 stores a three-dimensional image of the lymphatic vessels in the interior of a living body, acquired by a three-dimensional observation device, such as a CT apparatus. The three-dimensional image is acquired for example, by administering a contrast medium into the lymph fluid, and the lymphatic vessels are displayed as bright regions.

The projected-image generating circuit 104 generates the projected image G3 (see (a) in FIG. 3), which is associated with the fluorescence image G2 currently being acquired by the image-acquisition device 51, from the three-dimensional image stored in the three-dimensional image storage circuit 103, on the basis of the current position and current direction of the distal end of the inserted portion 2, which are input from the position sensor 4.

More concretely, when, for example, the operator inserts the distal end of the inserted portion 2 into the body via a hole formed in the surface of the body, in the state in which the distal end of the inserted portion 2 is disposed towards the inside of the hole at the entrance of the hole, the position and direction in this state are set as the reference position and reference direction. Also, in the three-dimensional image stored in the three-dimensional image storage circuit 103, the operator sets the position corresponding to the position of the hole and the insertion direction of the inserted portion 2 at the opening of the hole. By doing so, from the current position and current direction input from the position sensor 4, the image-acquisition position and image-acquisition direction of the fluorescence image G2 currently being acquired by the image-acquisition device 51 can be associated with the position and direction in the three-dimensional image.

Then, from the three-dimensional image, the projected-image generating circuit 104 extracts a three-dimensional space having an area corresponding to the acquisition region of the image-acquisition device 51 and having a prescribed size in the direction corresponding to the current direction of the inserted portion 2 and generates the two-dimensional projected image G3, which is formed by projecting the extracted three-dimensional image in the current direction of the inserted portion 2, that is, in the depth direction of the field of view. By doing so, the projected-image generating circuit 104 can generate a projected image G3 whose position is associated with the fluorescence image G2. In the generated projected image G3, pixels corresponding to the superficial lymphatic vessels A1 in the tissue and pixels corresponding to deep lymphatic vessels A2 in the tissue have the same degree of brightness values.

The multiplication processing circuit 105 generates the multiplied image G4 (see (c) in FIG. 3) by multiplying the brightness values of corresponding pixels in the fluorescence image G2 and the projected image G3 and displaying each pixel with a prescribed color having a luminance or hue according to the product obtained by multiplication. By doing so, in the multiplied image G4, the difference in luminance values between bright regions and dark regions common to both the fluorescence image G2 and the projected image G3 becomes larger, and the displayed observation targets common to both the fluorescence image G2 and the projected image G3 are displayed in an emphasized manner relative to the observation targets displayed only in the projected image G3. More concretely, regions where the lymphatic vessels A1 and A2 are displayed in both the fluorescence image G2 and the projected image G3, that is to say, regions corresponding to the superficial lymphatic vessels A1 in the tissue, are displayed in a deep or vivid color in the multiplied image G4. On the other hand, regions where the lymphatic vessels A1 and A2 are displayed in only one of the fluorescence image G2 and the projected image G3, that is to say, regions corresponding to the deep lymphatic vessels A2 in the tissue, are displayed in a light or pale color in the multiplied image G4. Therefore, the observer can more readily recognize the positions of the lymphatic vessels A1 and A2 in the depth direction, on the basis of the luminance or hue of each pixel in the multiplied image G4.

Here, the multiplication processing circuit 105 may perform any type of processing so that regions corresponding to the superficial lymphatic vessels A1 in the multiplied image G4 are displayed in a more emphasized manner than regions corresponding to the deep lymphatic vessels A2. For example, the multiplication processing circuit may perform processing for weighting the brightness values of the fluorescence image G2 by multiplying the brightness value of each pixel in the fluorescence image G2 by a prescribed coefficient, or by adding the prescribed coefficient thereto, and using the product or sum thereof in the multiplication processing. In addition, it may perform preprocessing, such as adjusting the tone curve of the fluorescence image G2 so as to sufficiently increase the difference in brightness/darkness between bright regions and dark regions in the fluorescence image G2.

Furthermore, the multiplication processing circuit 105 may perform processing for correcting the product to be within the appropriate range, so that the luminance or hue does not become saturated in the multiplied image G4 due to the product obtained by multiplying the brightness values of the fluorescence image G2 and the projected image G3 becoming too large.

The superposition processing circuit 106 generates the superposed image G5 (see (e) in FIG. 3) by superposing the multiplied image G4 generated by the multiplication processing circuit 105 on the white-light image G1 input from the white-light-image generating circuit 101. In other words, the superposed image G5 is an image in which the lymphatic vessels A1 and A2 in the white-light image G1 are associated with the morphology of the tissue B. The superposition processing circuit 106 outputs the generated superposed image G5 to the display controller 53.

Next, the operation of the endoscope system 1 including the thus-configured image-processing apparatus 100 will be described.

To observe tissue inside a living body, which is the subject X, using the endoscope system 1 according to this embodiment, the operator inserts the inserted portion 2 while alternately radiating white light and excitation light from the distal end of the inserted portion 2 by turning on the light source 31.

Then, when a lymphatic vessel A1 in a superficial layer of the tissue is present in the field of view acquired by the endoscope system 1, in the superposed image G5 displayed on the monitor 6, the lymphatic vessel A1 is displayed with a prescribed deep or vivid color. When a lymphatic vessel A2 is present at a comparatively deep position in the field of view, the lymphatic vessel A2 is displayed with a prescribed light or pale color. Of the lymphatic vessels A1 and A2 displayed in the superposed image G5, the observer performs the required treatment while ascertaining the three-dimensional structure of the lymphatic vessel A2 in a deep layer from portions whose color is light or pale, and distinguishing portions whose color is deep or vivid as superficial lymphatic vessels A1.

In this way, according to this embodiment, in the superposed image G5 shown to the observer, an image of superficial lymphatic vessels A1 in the tissue, which are considered to be of higher importance by the observer, is displayed in a more emphasized manner than deep lymphatic vessels A2 in the tissue, which are of lower importance. Thus, the observer can ascertain and get an overview of the three-dimensional structure of the lymphatic vessels A2 in deep layers while readily and accurately distinguishing the position of superficial lymphatic vessels A1 from the superposed image G5, and in addition, it is possible to prevent the superposed image G5 from becoming unnecessarily complicated for the observer.

In this embodiment, it has been assumed that lymphatic vessels A1 and A2 are the observation targets; instead of this, however, a plurality of observation targets may be observed. For example, in the case where a lesion is observed as an additional observation target, the lesion is tagged with a different fluorescent dye from the fluorescent dye used to tag the lymphatic vessels A1 and A2, and a three-dimensional image of the lesion is also stored in the three-dimensional image storage circuit 103. In this case, the multiplication processing circuit 105 displays the multiplied image G4 obtained from the fluorescence image G2 of the lymphatic vessels A1 and A2 and a multiplied image obtained from a fluorescence image of the lesion with a different appearance, for example, different colors. By doing so, two observation targets can be observed simultaneously while distinguishing between a surface layer and a deep layer.

To generate a fluorescence image and a multiplied image of a plurality of observation targets, a combination of different fluorescent dyes in which at least one of the excitation wavelength and the light-emission wavelength differ from each other is used, or alternatively, a combination of fluorescent dyes whose intensities at the light-emission wavelengths sufficiently differ is used.

In the former case, the illumination unit 3 radiates excitation light in a time-division manner, or the apparatus is configured to separate the light detected by the image-acquisition device 51 depending on the wavelength. The fluorescence-image generating circuit 102 should create separate fluorescence images for the plurality of observation targets, and the multiplication processing circuit 105 should use the individual fluorescence images in the multiplication processing.

In the latter case, the fluorescence-image generating circuit 102 creates a fluorescence image of the plurality of observation targets in the form of identical fluorescence images. The multiplication processing circuit 105 should create, for example, a histogram of the brightness values of the fluorescence image and should display each pixel group, in which the brightness values belong to two peaks appearing in the histogram, with different appearances.

In addition, regarding a lesion, the fluorescence image may be superposed directly on the white-light image without performing multiplication processing with the projected image.

In addition, it is possible to switch between displaying or not displaying a plurality of observation targets in the superposed image G5 based on an operation carried out by the observer. For example, using an input device (not illustrated), the operator selects and inputs one of a plurality of observation modes, and the superposition processing circuit 106 selects a multiplied image associated with the observation mode and creates a superposed image. By doing so, the observer can switch between displaying and not displaying the observation target in the superposed image G5 as needed.

In this embodiment, it has been assumed that a fluorescence image of lymphatic vessels is used as the superficial image; instead of this, however, a narrow-band light image of blood vessels may be used. In this case, the illumination unit 3 irradiates the subject X with blue narrow-band light and green narrow-band light instead of excitation light, and the three-dimensional image storage circuit 103 stores a three-dimensional image of the blood vessels. A narrow-band light image is an image in which capillary blood vessels in the surface layer of tissue and thick blood vessels at a comparatively deep position are displayed with high contrast, which enables observation of blood vessels as the observation target.

Moreover, although the multiplied image G4 is superposed on the white-light image G1 and shown to the observer in this embodiment, instead of this, the multiplied image G4 and the white-light image G1 may be shown to the observer in a juxtaposed manner.

In this embodiment, the image processing apparatus 100 may be provided in a separate unit from the endoscope system 1. In this case, the current position and current direction of the distal end of the inserted portion 2 inside the body are detected from outside the body by means of an X-ray observation apparatus or the like instead of the position sensor 4, and data on the detected current position and current direction are sent to the image processing apparatus 100 from the X-ray observation apparatus either wirelessly or via wires.

The display appearance of the multiplied image G4 in this embodiment is merely an example and can be freely modified. For example, a group of pixels whose products obtained by multiplying the brightness values in the multiplication processing circuit 105 are larger than a predetermined value may be surrounded with an outline, or this group of pixels may be displayed in a flashing manner on the superposed image G5.

In this embodiment, it has been assumed that images in which the lymphatic vessels A1 and A2 are both displayed as bright regions are used as the superficial image G2 and the projected image G3. Instead of this, however, a superficial image in which the lymphatic vessels are displayed as dark regions, like an infrared image, may be used; and in this case, multiplication processing with the projected image should be performed using a superficial image in which the brightness values are inverted.

### REFERENCE SIGNS LIST

1 endoscope system
2 inserted portion
21 objective optical system
22 first filter turret
3 illumination unit
31 light source
32 second filter turret
33 coupling lens
34 light guide fiber
35 illumination optical system
4 position sensor
5 control unit
51 image-acquisition device
52 timing controller
53 display controller
6 monitor
100 image processing apparatus, image processing unit
101 white-light-image generating circuit
102 fluorescence-image generating circuit
103 three-dimensional image storage circuit (storage unit)
104 projected-image generating circuit (projected-image generating unit)
105 multiplication processing circuit (multiplication processing unit)
106 superposition processing circuit (superposition processing unit)
A1 superficial lymphatic vessel
A2 deep lymphatic vessel
G1 white-light image
G2 fluorescence image (superficial image)
G3 projected image
G4 multiplied image
G5 superposed image
X subject

1. An image processing apparatus comprising:
a storage unit that stores a three-dimensional image of an observation target existing in a subject;
a projected-image generating unit that receives an image-acquisition position and an image-acquisition direction of a two-dimensional superficial image of the observation target in a surface layer of the subject and generates a two-dimensional projected image by projecting a position corresponding to the image-acquisition position of the three-dimensional image stored in the storage unit in the image-acquisition direction; and
a multiplication processing unit that receives the superficial image and the projected image generated by the projected-image generating unit and generates a multiplied image by multiplying brightness values of corresponding pixels in the superficial image and the projected image.

2. The image processing apparatus according to claim 1, wherein, in the multiplication, the multiplication processing unit uses a sum obtained by adding a coefficient to the brightness values of the superficial image or a product obtained by multiplying it by the coefficient.

3. The image processing apparatus according to claim 1, wherein the multiplication processing unit displays each pixel of the multiplied image with a luminance or hue according to the brightness value of each pixel.

4. The image processing apparatus according to claim 1, further comprising a superposition processing unit that receives a white-light image of the subject and generates a superposed image by superposing the multiplied image generated by the multiplication processing unit on the white-light image.

5. The image processing apparatus according to claim 4, wherein:
the multiplication processing unit uses images in which a plurality of observation targets are displayed as the superficial image and the projected image; and
the superposition processing unit superposes the plurality of observation targets on the white-light image using different display appearances.

6. The image processing apparatus according to claim 1, wherein the superficial image is a fluorescence image.

7. The image processing apparatus according to claim 1, wherein the superficial image is a narrow-band light image.
